(19) United States
(12) Patent Application Publication
(10) Pub. No.: US 2024/0311868 A1
(43) Pub. Date: Sep. 19, 2024

(54) DATABASE AND FILE MANAGEMENT SYSTEM FOR PREDICTIVE HEALTHCARE PRESCRIPTIONS

(71) Applicant: Harshit Jain, PARSIPPANY, NJ (US)

(72) Inventor: Harshit Jain, PARSIPPANY, NJ (US)

(21) Appl. No.: 18/672,923

(22) Filed: May 23, 2024

Related U.S. Application Data
(63) Continuation-in-part of application No. 18/379,056, filed on Oct. 11, 2023.
(63) Continuation-in-part of application No. 18/395,123, filed on Dec. 22, 2023.
(63) Continuation-in-part of application No. 18/436,013, filed on Feb. 7, 2024.
Application No. 18/395,123 is a continuation-in-part of application No. 18/116,290, filed on Mar. 1, 2023, now Pat. No. 11,869,673.
(63) Continuation-in-part of application No. 18/078,953, filed on Dec. 10, 2022.
(63) Continuation-in-part of application No. 17/857,013, filed on Jul. 3, 2022.

(51) Int. Cl. G06Q 30/0242; G06Q 30/0241; G06Q 30/0272

(52) U.S. Cl. CPC G06Q 30/0246; G06Q 30/0244; G06Q 30/0272; G06Q 30/0277

(57) ABSTRACT

The embodiments disclose a database and file management system including a digital platform configured to receive at least one advertiser digital initiative, a remote server configured to receive at least one advertiser digital initiative, a plurality of databases with a file management system configured to store known measurable metrics as files of at least one advertiser digital initiative message displayed for at least one health care provider, a processor configured to measure existing metrics of the at least one health care provider after the digital initiative message is displayed to the at least one health care provider and an intent to prescribe processor configured to calculate an intent to prescribe score based on comparing the known measurable metrics with existing the measured metrics and analyzing the comparison to determine a predetermined range of the intent to prescribe score for the measurable metrics for use by the advertiser.

### BACKGROUND

In the majority of the markets across the globe, it is a challenging task for digital marketers to access e-prescription data and data around their digital endeavors. The efforts of medical representatives, digital campaigns, e-platform activations, and similar initiatives often go unnoticed, lacking quantification and identification as crucial campaign metrics.

As a result, digital marketers are unable to assess and so credit the consequences of their efforts in any way to campaign-specific criteria. Digital marketers have no idea what is driving their initiatives to succeed or fail, and there is no solid basis for leveraging and utilizing the capabilities of digital media as it should be.

The absence of campaign attribution leaves stakeholders in the digital landscape including e-marketers, advertisers, and agencies unable to optimize or expand the scope of their digital marketing efforts. Campaigns are created and run without the benefit of actual business metrics.

### BRIEF SUMMARY OF THE INVENTION

The present invention is a database and file management system and platform for predictive healthcare prescribing. The database and file management system and platform includes a digital marketing campaign solution that can evaluate campaign outcome effectiveness, which will be referred to herein as an Intent to Prescribe Score (I2P Score). In addition, combined with the I2P Score is a framework of campaign metrics, aimed at gauging the intent or willingness of Healthcare Professionals (HCPs) to prescribe a particular drug formulation during a specific brand campaign. This approach involves observing HCPs behavioral patterns, particularly their engagement with advertisements and consumption of the advertisement message, to calculate the I2P score effectively.

By utilizing contemporary digital marketing campaign criteria such as message affinity score, message frequency, and message view time, among others, the prescription behavior of HCPs is evaluated. The I2P Framework evaluation leads to the derivation of the I2P Score and its attribution to campaign performance. Consequently, digital marketers and other stakeholders in healthcare AdTech worldwide have access to a dependable standard for assessing the contribution of campaign parameters to their campaign outcomes.

The I2P Framework represents an innovative benchmark designed to empower stakeholders within the digital marketing landscape. Stakeholders benefit, particularly those operating in the pharmaceutical sector and that are actively seeking a benchmark to address the challenge of measuring HCP behavior regarding prescription intent.

### BRIEF DESCRIPTION OF THE DRAWINGS

FIG. 1 shows for illustrative purposes only an example of an I2P framework of one embodiment.

FIG. 2 shows a block diagram of an overview flowchart assessing the prescription behavior of HCPs of one embodiment.

FIG. 3 shows for illustrative purposes only an example of measurable metrics of one embodiment.

FIG. 4 shows for illustrative purposes only an example of a digital platform of one embodiment.

FIG. 5 shows for illustrative purposes only an example of an I2P mechanism of one embodiment.

FIG. 6 shows for illustrative purposes only an example of measuring the viewability of one embodiment.

FIG. 7 shows for illustrative purposes only an example of measuring the engagement of one embodiment.

FIG. 8 shows for illustrative purposes only an example of publisher platform types of one embodiment.

FIG. 9 shows a block diagram of an overview of an example of an I2P score formula of one embodiment.

### DETAILED DESCRIPTION OF THE INVENTION

In the following description, reference is made to the accompanying drawings, which form a part hereof, and in which is shown by way of illustration a specific example in which the invention may be practiced. It is to be understood that other embodiments may be utilized and structural changes may be made without departing from the scope of the present invention.

### General Overview

It should be noted that the descriptions that follow, for example, in terms of an intent to prescribe score (I2P Score) system, are described for illustrative purposes and the underlying system can apply to any number and multiple types of database and file management systems, digital platforms and website advertisers. In one embodiment of the present invention, the intent to prescribe (I2P) is a scoring system that can be configured using HCP behavior related to the messaging of advertising medical products. The intent to prescribe score system can be configured to include databases and file management systems and platforms to store HCP behaviors and can be configured to include measurable metrics using the present invention.

For example, message utilization presents a notable challenge for advertisers. Aligning the appropriate commercial message with the relevant Healthcare Professional (HCP) is a complex and time-consuming task. For example, extensive research is conducted to ensure that an advertisement containing information about cardiology-related medical products reaches an HCP specializing in cardiology rather than one in a different medical field, such as dentistry or orthopedics. In the future, healthcare and life sciences marketers will increasingly require access to information about recent and innovative medical solutions, enabling them to effectively reach the corresponding HCPs and medical practitioners on a large scale.

Furthermore, a substantial amount of medical product content, authored by experts in the medical field, is intended solely for consumption by specialist medical practitioners. In such instances, an authorization mechanism plays a role in restricting public access to this medical product content. Moreover, advertisers may limit access to detailed medical product content exclusively to pre-identified HCPs, often tailoring the experience for HCPs visiting specific sections or pages on their websites.

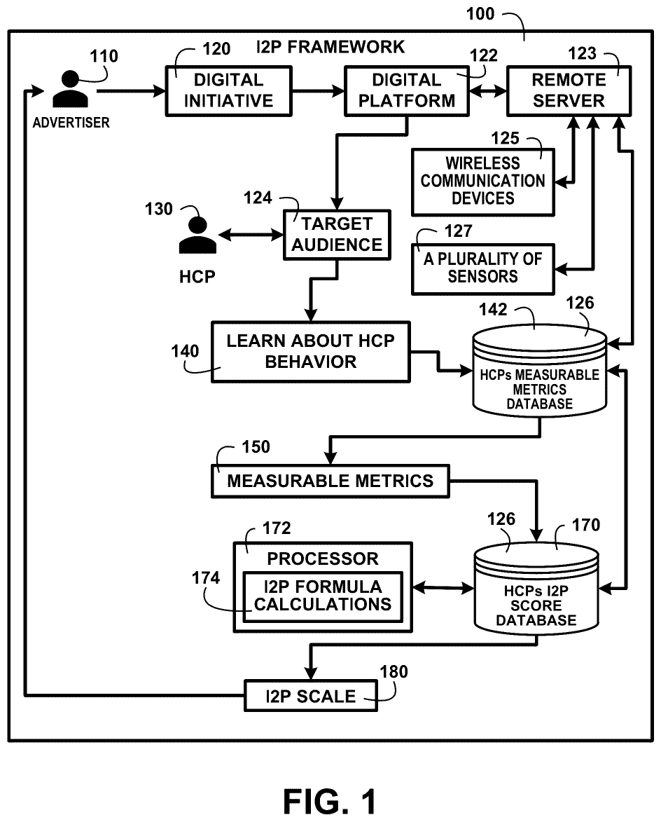

FIG. 1 shows a block diagram of an overview of the present invention with a database and file management system and platform for predictive healthcare prescribing with an I2P framework of one embodiment. FIG. 1 shows an I2P framework 100 comprising an advertiser 110 advertising a digital initiative 120 for advertising medical products including pharmaceutical products on a digital platform 122. The digital platform 122 has a remote server 123 coupled to a plurality of databases 126, at least one processor 172, wireless communication devices 125, and a plurality of sensors 127.

The digital initiative 120 is directed to a target audience 124 of health care professionals (HCPs). The digital initiative 120 is displayed on the digital devices of an HCP 130 after the advertiser 110 has received authorization from the HCP 130. Relationships between the parties are described in U.S. Ser. No. 18/436,013 filed Feb. 7, 2024 which is incorporated herein by reference, and U.S. Ser. No. 18/395,123 filed Dec. 22, 2023 which is incorporated herein by reference, and U.S. Ser. No. 18/116,290 filed Mar. 1, 2023, which is incorporated herein by reference.

The advertiser 110 can access data from the digital platform 122 to learn about HCP behavior 140 related to the messaging of the advertising medical products. The aspects of the HCP behavior data are stored on an HCPs measurable metrics database 142.

The stored data are measurable metrics 150 which are processed to determine an intent to prescribe (I2P) score. The analysis of the measurable metrics 150 includes an I2P formula to calculate the I2P score. The resulting I2P scores of the advertisement messaging are stored on an HCPs I2P score database 170. The analysis uses a processor 172 I2P and formula calculations 174 are ranked on an I2P scale 180. The I2P score rankings for the message related to the ads are available to the advertiser 110.

An I2P Score serves as a user-friendly and unique tool that organizations can utilize to discern measurable performance metrics of their online advertising campaigns and digital initiatives. This tool is particularly beneficial for marketers in regions where establishing correlations between digital campaigns and quantifiable metrics is generally challenging.

The I2P Score primarily calculates various campaign parameters and assigns a measurable quantity or score to the prescription behavior of Healthcare Professionals (HCPs) targeted by a campaign. Specifically, the I2P Score framework takes into account established campaign metrics and markers, integrating them into a precise formula that predicts the “Intent to Prescribe” score or the I2P Score for healthcare professionals who have viewed or interacted with an online campaign.

The ability to predict the prescription intent of HCPs enables decision-makers in the digital marketing domain to improve their campaign outcomes and other digital initiatives. For instance, a low I2P Score indicates underperformance of campaigns, possibly due to ineffective advertising channels or inadequate advertisement frequency. Armed with this critical information, marketers can review and adjust their choice of channels or modify the frequency of serving advertisement messages to HCPs, thus improving the outcomes of their campaigns accordingly.

### Detailed Description:

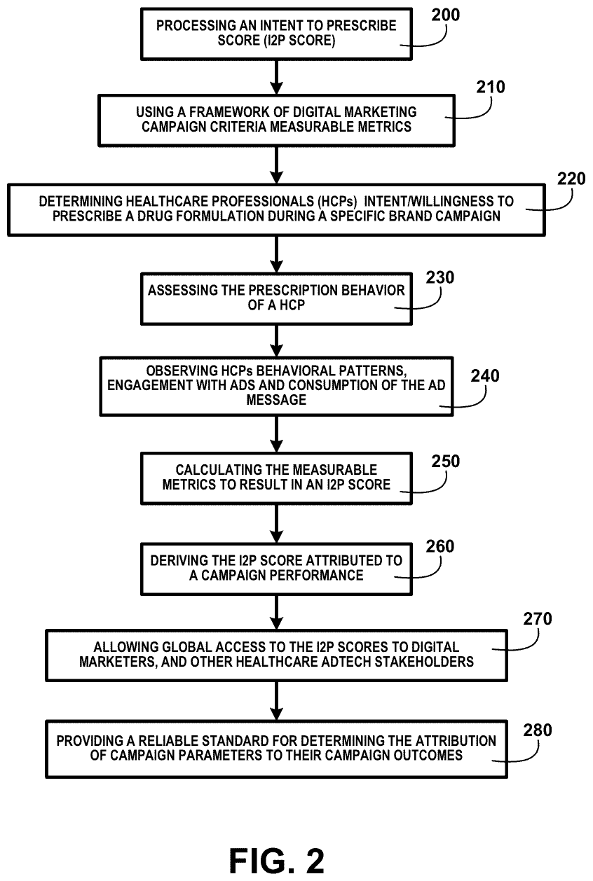

FIG. 2 shows a block diagram of an overview flowchart assessing the prescription behavior of HCPs of one embodiment. FIG. 2 shows processing an intent to prescribe score (I2P score) 200 using a framework of digital marketing campaign criteria measurable metrics 210. Determining healthcare professionals' (HCPs) intent/willingness to prescribe a drug formulation during a specific brand campaign 220. Assessing the prescription behavior of a HCP 230.

Observing HCPs behavioral patterns, engagement with advertisements, and consumption of the advertising message 240. Calculating the measurable metrics to result in an I2P score 250. Deriving the I2P score attributed to a campaign performance 260. Allowing global access to the I2P scores to digital marketers, and other healthcare AdTech stakeholders 270. Providing a reliable standard for determining the attribution of campaign parameters to their campaign outcomes 280.

Obtaining the I2P Score is based on multiple campaign metrics and markers, the I2P Score determines how HCPs perceive the advertisement-based information that is shared with them. The I2P scoring mechanism primarily considers two key aspects of the HCP behavior 140 of FIG. 1: first, how they view these messages (viewability), and second, how they interact with the messages they receive (engagement). The I2P mechanism includes two aspects of the HCP behavior 140 of FIG. 1 in detail and how they affect and influence the overall 12P Score.

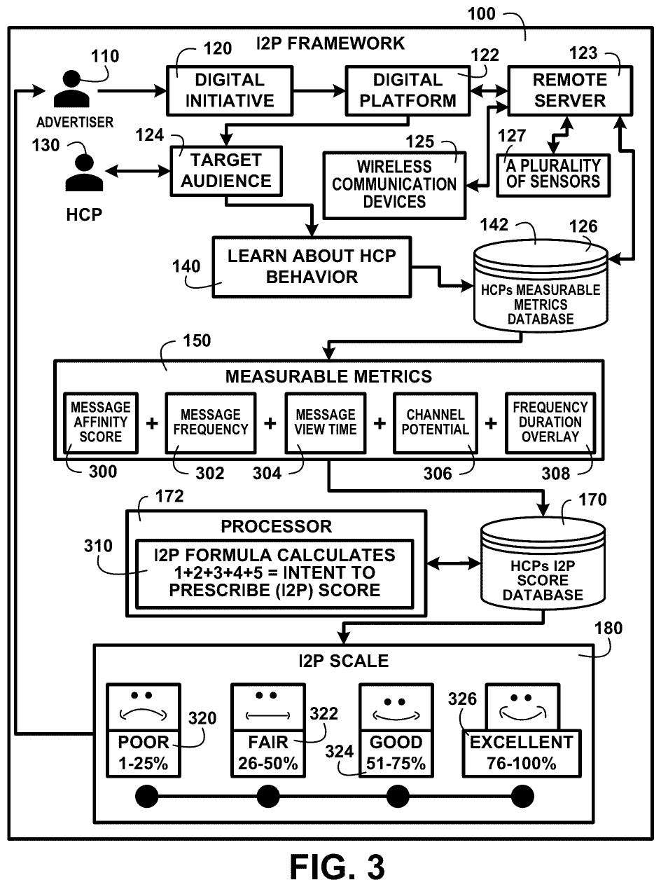

FIG. 3 shows for illustrative purposes only an example of measurable metrics of one embodiment. FIG. 3 shows the I2P framework 100 to rank metrics of an advertiser 110 digital initiative 120 being displayed on a digital platform 122. The digital platform 122 has a remote server 123 coupled to a plurality of databases 126, at least one processor 172, wireless communication devices 125, and a plurality of sensors 127. The advertiser 110 digital initiative 120 is being accessed by a target audience 124. Relationships between the parties are described in U.S. Ser. No. 18/436,013 filed Feb. 7, 2024 which is incorporated herein by reference, and U.S. Ser. No. 18/395,123 filed Dec. 22, 2023 which is incorporated herein by reference, and U.S. Ser. No. 18/116,290 filed Mar. 1, 2023, which is incorporated herein by reference.

The target audience 124 is made up of an HCP 130 working on a patient and seeing advertisements being displayed on the HCP's mobile device using a point of care application and/or electronic health record application. The HCP 130 has previously given their authorization for the advertiser 110 to display advertisements and other information typically related to the specialty of the HCP 130. The I2P framework 100 is used to learn about HCP behavior 140. The HCP behavior 140 is collected and stored on an HCPs measurable metrics database 142.

The measurable metrics 150 include message affinity score 300, message frequency 302, message view time 304, channel potential 306, and frequency duration overlay 308. The measurable metrics 150 are stored in an HCPs I2P score database 170. A digital processor 172 using an I2P formula calculates 1+2+3+4+5=intent to prescribe (I2P) score 310. The I2P scale 180 is used to report the I2P score in four rankings including poor 1-25% 320, fair 26-50% 322, good 51-75% 324, and excellent 76-100% 326. The I2P score of the HCP behavior is accessible to the advertiser 110 to assist in evaluating the advertisement campaign attributions of the advertisement messaging.

Platform Agnostic: The I2P Score utilizes widely accepted industry standards, making the entire 12P framework applicable to and usable by all campaigns targeting Healthcare Professionals (HCPs) across any platform frequented by them. This framework seamlessly adapts to various HCP-accessible platforms such as online medical journals, Electronic Health Record (EHR) systems, telemedicine platforms, e-prescribing platforms, physician networking platforms, and online learning portals. Especially beneficial for marketers and advertisers in regions worldwide where assessing the effectiveness of digital campaigns targeting HCP audiences is challenging.

Built on Real-World Experiences: The formula employed by the I2P framework is backed by substantial real-world experiences and practical use cases. Its application in multiple scenarios not only enhances the credibility of the framework but also offers empirical evidence of its accuracy in successfully predicting campaign outcomes.

Improvement Indicator: The I2P Score consolidates diverse industry standards, meaning even a slight alteration in the constituent marker or metric directly impacts the final calculated I2P Score value. As a comprehensive framework encompassing crucial markers, the I2P Score derived for a campaign enables users to evaluate the performance of different metrics and adjust underperforming metrics to achieve optimal outcomes.

Moreover, while the I2P Score serves as a tool for gaining insights into current campaign performance, it also functions as a reliable indicator for managing and enhancing overall campaign strategy. For instance, users may identify a need to increase advertisement frequency to improve their campaign's I2P Score, thus making it a pivotal tool for users to refine their business strategy and consistently achieve desired objectives.

Benefits of I2P: The I2P framework empowers marketing and businesses by addressing real-world challenges and driving tangible impact. Grounded in rich campaign data and real-life use cases, the framework employs standard industry markers and metrics, assigning appropriate weightage based on their correlation. Consequently, marketers and businesses utilizing this framework can analyze the I2P Score and take corrective measures to improve business outcomes.

The I2P framework represents a significant shift, moving business strategy from relying on fragmented data to acquiring actionable insights, thereby fostering enhanced outcomes. In an era dominated by digital adoption, the I2P framework provides a potential tool for users whose success hinges on understanding behavioral changes among HCPs to outperform competitors consistently.

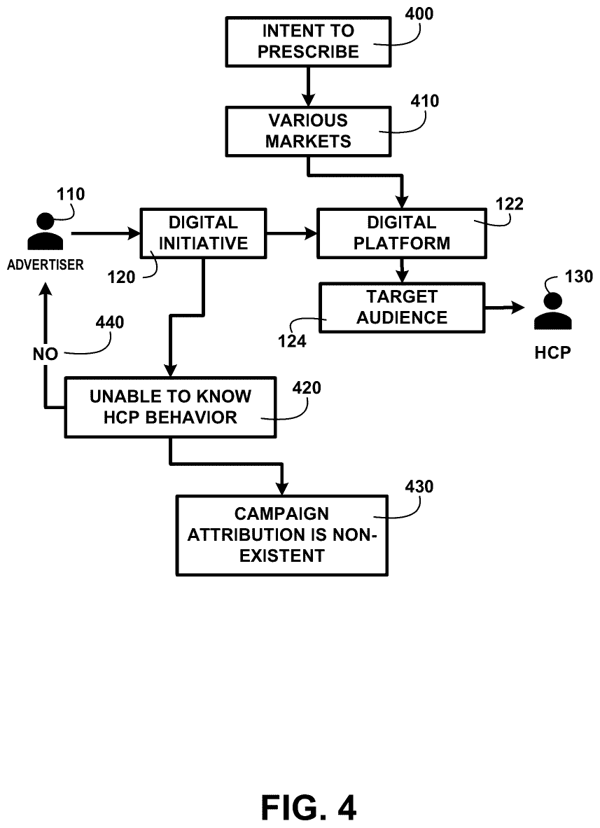

FIG. 4 shows for illustrative purposes only an example of a digital platform of one embodiment. FIG. 4 shows an intent to prescribe 400 evaluations of at least one HCP 130 to prescribe advertised medical products in various markets 410. The advertised medical products are displayed on an HCP 130 mobile device by the digital platform 122. The digital platform 122 displays the advertised medical products through an advertiser 110 submitted digital initiative 120.

Although the HCP 130 has authorized the displaying of the advertiser's advertised medical products, in the various markets 410, for example, sufficient technology may not be available for the advertiser 110 to directly access the attributes of the HCP behavior related to the advertising including e-prescribing, medical product messaging from the digital platform 122 displays to the target audience 124 for that advertising campaign. Unable to know HCP behavior 420 directly from HCP 130 behavior and only receiving a query response of no 440 leads to the reality that the campaign attribution is non-existent 430. The intent to prescribe score system is the alternative solution to the outside circumstances that create the problem of one embodiment.

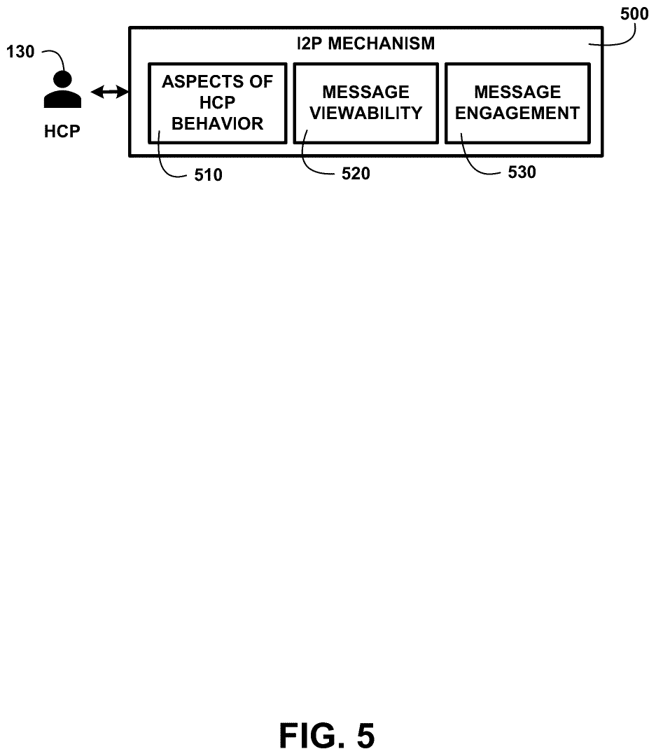

FIG. 5 shows for illustrative purposes only an example of the I2P mechanism of one embodiment. FIG. 5 shows the relationship of an HCP 130 interaction with a message as an I2P mechanism 500 in obtaining an I2P score. The HCP 130 interaction with a message is used to measure aspects of HCP behavior 510 related to messaging in an advertiser's advertisement campaign for a medical product. In one embodiment, message viewability 520 and message engagement 530 are measured to quantify aspects of HCP behavior 510 related to the messaging.

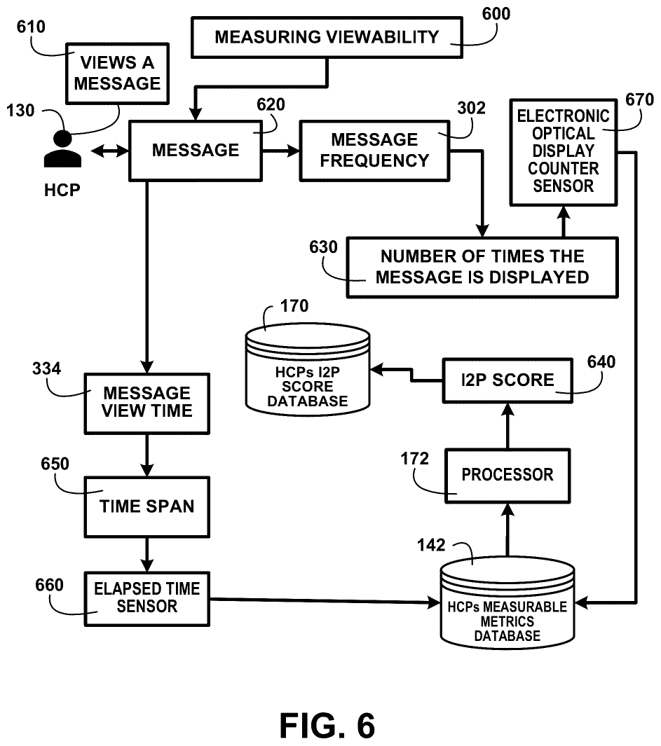

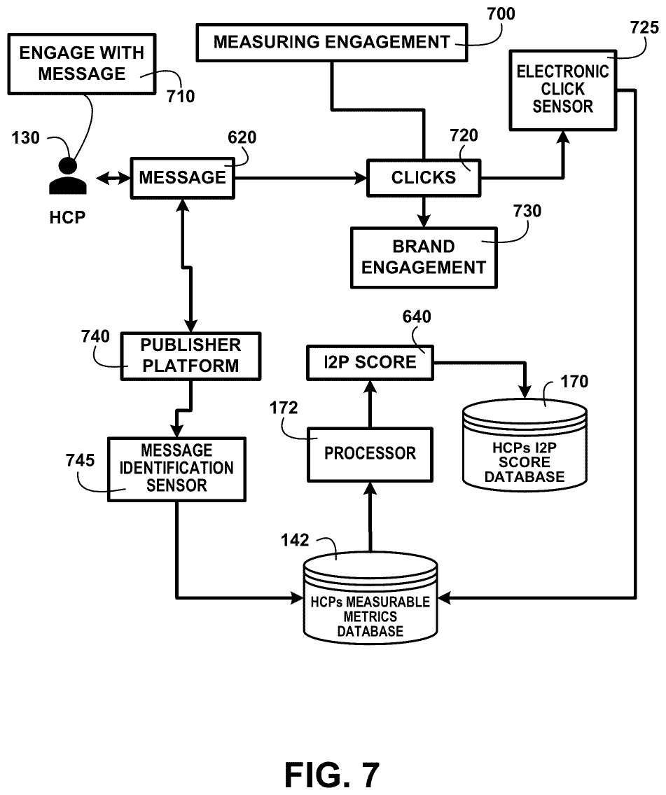

Based on multiple campaign metrics and markers, the I2P Score determines how HCPs perceive the advertisement-based information that is shared with them. The I2P scoring mechanism primarily considers two key aspects of the HCP behavior. First, how they view these messages (viewability). Second, how the HCP 130 interacts with the messages they receive (engagement). FIG. 6 and FIG. 7 describe these two aspects of the HCP behavior in detail and how they affect and influence the overall 12P Score.

FIG. 6 shows for illustrative purposes only an example of measuring the viewability of one embodiment. FIG. 6 shows measuring viewability 600 as an HCP 130 views a message 610. Measuring viewability 600 for an advertiser's message 620 includes message frequency 302. Message frequency 302 is the number of times the message is displayed 630. An electronic optical display counter sensor 670 transmits the number of times to the HCPs measurable metrics database 142. An elapsed time sensor 660 measuring the time span 650 and transmitting the elapsed time to the HCPs measurable metrics database 142. The processor 172 calculates the I2P score value of the metrics measurements and stores the results in the HCPs I2P score database 170.

The number of times the message is displayed 630 is factored into the I2P score 640. The message view time 334 is the time span 650 for which the HCP 130 views a message 610. The message view time 334 is also a factor in determining the I2P score 640.

Viewability is a marker that indicates the degree of message consumption for a given campaign. The overall viewability of a displayed message is in itself based on two key variables namely: 1. Frequency, which represents the number of times a message has been displayed to a unique HCP during a campaign, and 2. View Time, which indicates the duration or time span for which the message is viewed by a unique HCP.

In this way message viewability has the potential to influence the awareness and consequently the attitude of the HCP, which is an important outcome for any online marketer to know and utilize it optimizing their campaigns.

FIG. 7 shows for illustrative purposes only an example of measuring the engagement of one embodiment. FIG. 7 shows measuring engagement 700 as measured when an HCP 130 will engage with message 710 displayed by a publisher platform 740. The HCP 130 views the message 620 and may perform clicks 720 to see more information on the products which is referred to as brand engagement 730. The number of clicks 720 is a component of an I2P score 640 for that specific campaign. The publisher platform 740 displaying the message is also factored into the I2P score 640.

An electronic click sensor 725 transmits the number of times to the HCPs measurable metrics database 142. A message identification sensor 745 identifies each message by its unique ID number and transmits the message identification to the HCPs measurable metrics database 142. The processor 172 calculates the I2P score value of the metrics measurements and stores the results in the HCPs I2P score database 170.

The way an HCP 130 interacts or engages with a message determines the degree of interest they have or show in that particular message and impacts the measuring engagement 700 score component of the I2P score 640. Two aspects of engagement are of importance from the I2P Score's point of view.

First, there are the clicks, which in the digital marketing domain measures the interaction between brands and HCPs. When the HCP 130 clicks 720 on a message they are in a way expressing their interest in that particular product or service promoted by the respective brand—this also reinforces the relevance of the message to the target audience 124 of FIG. 1 (HCPs) and moreover indicates the empathy shown by the HCP towards the message that is viewed and subsequently clicked by them.

Second, apart from the clicks, another crucial factor that is considered as part of end-user (HCP) engagement is the type of publisher platform 740 the HCP is making use of at that particular time. In simple terms the weightage assigned to the quality of engagement varies by the type of platform the HCP is using during the campaign. The engagement of the HCP with an advertising message over certain publisher platforms is given more weightage in the calculation of the I2P Score compared with other publisher platforms. Here it is important to have a deeper understating of the different publisher platforms that are factored in the calculation process for arriving at an I2P Score for a campaign and the weightage that is assigned to the different publisher platform types.

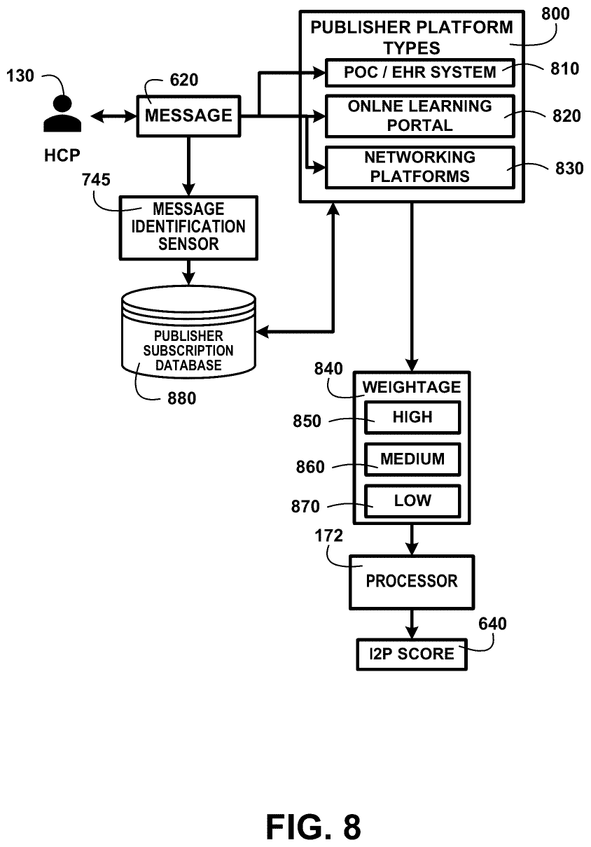

FIG. 8 shows for illustrative purposes only an example of publisher platform types of one embodiment. FIG. 8 shows an HCP 130 view of a publisher platform message 620. The publisher platform types 800 are used as a weighting factor in the determination of an I2P score. The types of publisher platforms include a point of care (POC) and electronic health record (EHR) system POC/EHR system 810, and an online learning portal 820 and networking platforms 830. The weightage 840 of the publisher platform types 800 is assigned as high 850, medium 860, and low 870, and then applied to the I2P score 640.

The message includes an identification number that also identifies the publisher platform type. The message identification sensor 745 transmits the message identification to the publisher subscription database 880. The publisher subscription database 880 identifies the publisher platform type which is forwarded to the processor 172 to determine the weightage 840 for use in calculating the I2P score 640.

During a working week, HCPs access various publisher platforms to do diverse professional and personal activities. For example, they would access the Telehealth or EHR (Electronic Health Record) system for professional reasons and may switch over to Online Learning Portals or websites where Online Medical Journals are available, for the purpose of learning. They may also access the Online Medical Association websites for connecting with their fellow colleagues or acquaintances within the medical fraternity. Thus, within a span of a few hours, on a given day, HCPs may switch from one publisher platform to another for specific purposes.

Following are the categories of publisher platforms that an HCP may access and are considered in the calculation of the I2P Score: 1. Telehealth platforms/EHRs (for providing virtual consultation to their patients in case of telehealth platforms/apps and in-person consultation in case of face-to-face sessions over Point of Care (POC) systems), 2. Online Learning Portals/Online Medical Journals (for seeking information about the latest happenings in the healthcare and life sciences domain such as new drug launches, new therapies, new breakthroughs, innovative medical technologies, etc.), and 3. Networking Platforms/Online Medical Associations (for connecting with peers and acquaintances within the medical community).

Publisher platforms considered within the I2P framework are selected based on historical data collected from past campaigns. The weighting or preference assigned to these platform types in the I2P Score framework follows a hierarchy from highest to lowest weightage: Telehealth platforms/EHRs>Online Learning Portals/Online Medical Journals>Networking Platforms/Online Medical Associations. Consequently, activities of Healthcare Professionals (HCPs) on Telehealth platforms/EHRs carry more weight than those on platforms like Online Learning Portals/Online Medical Journals, with activities on Networking Platforms/Online Medical Associations given the least weight of all. The I2P Score serves as an effective and useful metric for evaluating the outcomes of digital marketing campaigns for several reasons. Key characteristics that make the I2P Score a potent digital marketing metric include:

Pertinent Markers: The markers used to derive the I2P Score adhere to industry standards such as message affinity, message frequency, view time, and Channel Potential 306 (as illustrated in FIG. 3). While these standards are commonly employed individually to assess various aspects of a campaign, their integration for calculating the I2P Score unlocks its full potential. This is particularly advantageous for marketers and advertisers operating in global markets where measuring the outcome of digital campaigns, especially within the healthcare and life sciences domains, is challenging.

Smart Weightages: Each marker utilized in calculating the I2P Score is assigned an appropriate weightage. The weightage assigned to each marker reflects its contribution to the overall probabilistic score. For instance, actions taken by HCPs on Telehealth platforms/EHRs are weighted more heavily than activities on Online Learning Portals/Online Medical Journals or Networking Platforms/Online Medical Associations. This allocation of weightage provides users with a clear understanding of a campaign's true effectiveness. In the aforementioned scenario, HCPs targeted within a campaign are more likely to prescribe if their activities predominantly occur on Telehealth platforms/EHRs compared to other platforms such as Online Learning Portals/Online Medical Journals or Networking Platforms/Online Medical Associations.

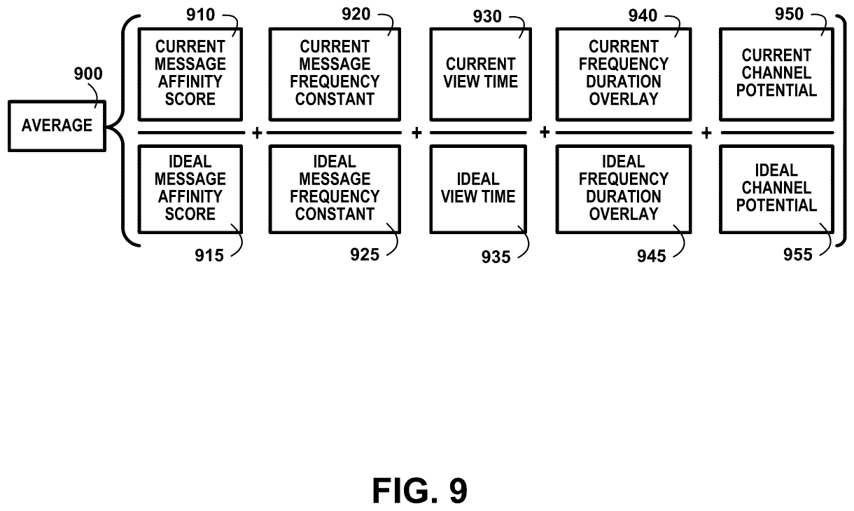

FIG. 9 shows for illustrative purposes only an example of the I2P score of one embodiment. FIG. 9 shows an average of 900 of the percentage results of calculation that divide current campaign metrics scores by ideal campaign metrics scores. The campaign measurable metrics scores are the quotients of the following pair of scores including a current message affinity score 910 divided by an ideal message affinity score 915; a current message frequency constant 920 divided by an ideal message frequency constant 925; a current view time 930 divided by an ideal view time 935; a current frequency duration overlay 940 divided by an ideal frequency duration overlay 945; and a current channel potential 950 divided by an ideal channel potential 955.

The I2P Score provides a universal parameter that takes into consideration all relevant digital marketing metrics such as viewability and engagement of an existing campaign and fine-tunes these metrics at a hyper level to realistically calculate the performance of any campaign.

The I2P Formula includes I2P metrics that are rated, and then an average of the quotient ratings results in an I2P Score. The following lists various campaign metrics factored in for calculating the I2P Score for any digital campaign showing the Metric followed by a description of the Metric.

Unique Reach: This metric refers to the total number of unique HCPs who have been reached via a particular digital campaign or the total number of unique HCPs who saw the message.

Impression: The number of times the message or advertisement is displayed to an HCP is referred to as an impression. More impressions typically lead to a higher inclination to act.

Click: HCP clicking on a display banner demonstrates a higher affinity of relevance to the message. Naturally, more clicks mean more engagement.

Message Affinity: It is a measure of perception lift among the HCPs before and after the campaign.

Channel Affinity: It is the affinity that the HCPs feel towards a particular channel for a particular kind of message. It is important to select the right channels for any messaging or campaign to ensure greater uptake of the message.

Frequency: The number of times a message is displayed to an HCP within a given time period is measured as frequency. It is a critical factor in building awareness and resonance.

View Time: It is a particularly important score that tells us about the time that HCPs spent looking at or reading the message. It shows the importance and relevance HCPs assign to that message.

Frequency and Duration Overlay: It is a particularly important score that tells us about the time that HCPs spent looking at or reading the message. It shows the importance and relevance HCPs assign to that message.

The I2P formula comprises five major components as follows: Message Affinity, Score 300 of FIG. 3, Message Frequency 302 of FIG. 3, Message View Time 304 of FIG. 3, Channel Potential 306 of FIG. 3, and Frequency Duration Overlay 308 of FIG. 3. The following is a description of each of the components that goes into the calculation of the I2P Score:

Message Affinity—This particular component represents the measure of brand or campaign recall by the targeted audience (HCPs) via a Brand Lift Study (BLS) and/or clicks (click-through rate or CTR). Both the subcomponents, BLS and CTR are given equal weightage in the calculation of the Message Affinity component.

Message Frequency—Also referred to as the Message Affinity Constant within the I2P framework 100 of FIG. 1 represents a constant that comprises the number of times an individual from the targeted audience (HCPs) saw a message and the total audience that saw the said message, during a campaign. Here again, there are five different frequency bands where each frequency band or range is assigned a specific weight. The higher the Message Frequency band/range, the higher the weightage assigned to it.

Message View Time—This particular component of the I2P Score represents the Total Time (in seconds) that a particular individual from the target audience 124 of FIG. 1 spent in viewing the message that was sent across during a particular campaign.

Channel Potential 306 of FIG. 3—This represents the Channel or Platform Category over which an individual member of the targeted audience viewed the message during the said campaign. The Channel Potential 306 of FIG. 3 is categorized as the Point-Of-Care (POC) channel, the Endemic channel, and the General channel. Here again, distinct categories are assigned different weights in the following order: POC channel (having the highest weightage), followed by Endemic channel (having a weight that is lesser than the POC channel and greater than the General channel), and the final subcomponent here is the General channel (having the lowest weightage assigned to it).

Frequency Duration Overlay 308 of FIG. 3—This particular component of the I2P framework 100 of FIG. 1 represents the length of the campaign against frequency that effectively establishes the TOM (top of mind) recall. The TOM in digital marketing parlance describes the first brand that comes to the mind of the targeted audience when they think about a specific industry or line of business.

Analyzing the I2P Score is performed once data is available against the required metrics the I2P Score is derived accordingly. The likelihood that the targeted group of HCPs for a particular digital advertising campaign will prescribe the product being advertised in the campaign (or intend to do so) is represented by the I2P Score.

The I2P Score factors in key digital marketing standards such as the count of impressions received by the targeted audience during the campaign, the CTR for the campaign, view time of the targeted audience, etc.

Furthermore, the I2P Score also factors specific pharma related such as HCP perception lift (Message Affinity) and HCP Channel Potential 306 (Point-of-Care, Endemic, or General) that specify how close or far the targeted HCPs are from prescribing the product or service being promoted by the brand campaign.

The following describes what the I2P Score represents showing the Indicator; I2P Score; and Score Description.

Excellent; 76%-100%; When a campaign has this rating, it means that the marketer or advertiser is conducting all campaign-related tasks effectively. The advertising is performing well (an excellent campaign).

Good; 51%-75%; When a campaign obtains this specific grade, it means that its performance is on par with most other campaigns and that the advertiser/marketer may anticipate a good return on investment (ROI) for the money spent (a good campaign).

Fair; 26%-50%; When a campaign obtains this specific score, it indicates that there is more room for optimization and enhancement to increase its effectiveness beyond its current state (a fair campaign).

Poor; 1%-25%; When a campaign gets this specific score, it means that the campaign is not performing well at all. Moreover, the concerned marketer/advertiser immediately needs to address the shortcomings and make necessary modifications to augment the effectiveness of the campaign (an ineffective campaign).

The foregoing has described the principles, embodiments, and modes of operation of the present invention. However, the invention should not be construed as being limited to the particular embodiments discussed. The above-described embodiments should be regarded as illustrative rather than restrictive, and it should be appreciated that variations may be made in those embodiments by workers skilled in the art without departing from the scope of the present invention as defined by the following claims.

What is claimed is:

1. A database and file management system, comprising:
a digital platform having at least one wireless communication device configured to receive at least one advertiser digital initiative;
a remote server coupled to the digital platform configured to receive at least one advertiser digital initiative;
a plurality of databases with a file management system coupled to the remote server configured to store known measurable metrics as files of at least one advertiser digital initiative message displayed for at least one health care provider;
a processor configured to measure existing metrics of the at least one health care provider after the digital initiative message is displayed to the at least one health care provider; and
an intent to prescribe processor configured to calculate an intent to prescribe score based on comparing the known measurable metrics with existing the measured metrics and analyzing the comparison to determine a predetermined range of the intent to prescribe score for the measurable metrics for use by the advertiser to assess the efficacy of the messaging attributes.

2. The database and file management system of claim 1, wherein the intent to prescribe calculation comprises five major components including a message affinity score, message frequency, message view time, channel potential, and frequency duration overlay.

3. The database and file management system of claim 1, wherein the predetermined range of the intent to prescribe score includes classifications as poor in a range from 1% to 25%, fair in a range from 26% to 50%, good in a range from 51% to 75%, and excellent in a range from 76% to 100%.

4. The database and file management system of claim 1, further comprising campaign measurable metrics scores as quotients of a following pair of scores.

5. The database and file management system of claim 1, further comprising a view time measurable metric which indicates a time span for which the message is viewed by a unique health care provider.

6. The database and file management system of claim 1, further comprising a message frequency measurable metric which is a number of times the message is displayed.

7. The database and file management system of claim 1, further comprising a plurality of sensors coupled to the remote server configured to measure the measurable metrics.

8. A database and file management system, comprising:
a digital platform having at least one wireless communication device configured to receive at least one advertiser digital initiative;
a remote server coupled to the digital platform configured to receive at least one advertiser digital initiative;
a plurality of databases with a file management system coupled to the remote server configured to store known measurable metrics as files of at least one advertiser digital initiative message displayed for at least one health care provider;
a processor configured to measure existing metrics of the at least one health care provider after the digital initiative message is displayed to the at least one health care provider;
an intent to prescribe processor configured to calculate an intent to prescribe score based on comparing the known measurable metrics with existing the measured metrics and analyzing the comparison to determine a predetermined range of the intent to prescribe score for the measurable metrics for use by the advertiser to assess the efficacy of the messaging attributes; and
a view time measurable metric which indicates the time span for which the message is viewed by a unique health care provider.

9. The database and file management system of claim 8, wherein the intent to prescribe calculation comprises five major components including a message affinity score, message frequency, message view time, channel potential, and frequency duration overlay.

10. The database and file management system of claim 8, wherein the predetermined range of the intent to prescribe score includes classifications as poor in a range from 1% to 25%, fair in a range from 26% to 50%, good in a range from 51% to 75%, and excellent in a range from 76% to 100%.

11. The database and file management system of claim 8, further comprising campaign measurable metrics scores are quotients of a following pair of scores.

12. The database and file management system of claim 8, further comprising a message frequency measurable metric which is a number of times a message is displayed.

13. The database and file management system of claim 8, further comprising a plurality of sensors coupled to the remote server configured to measure the measurable metrics.

14. The database and file management system of claim 8, wherein the digital platform is a web server coupled to a local device and configured to display the one advertiser digital initiative message to the at least one health care provider.

15. A database and file management system, comprising
a digital platform having at least one wireless communication device configured to receive at least one advertiser digital initiative;
a remote server coupled to the digital platform configured to receive at least one advertiser digital initiative;
a plurality of databases with a file management system coupled to the remote server configured to store known measurable metrics as files of at least one advertiser digital initiative message displayed for at least one health care provider;
a processor configured to measure existing metrics of the at least one health care provider after the digital initiative message is displayed to the at least one health care provider;
a web server coupled to digital platform, remote server and a local device and configured to display the one advertiser digital initiative message to the at least one health care provider
an intent to prescribe processor configured to calculate an intent to prescribe score based on comparing the known measurable metrics with existing the measured metrics and analyzing the comparison to determine a predetermined range of the intent to prescribe score for the measurable metrics for use by the advertiser to assess the efficacy of the messaging attributes; and
a view time measurable metric which indicates the time span for which the message is viewed by a unique health care provider.

16. The database and file management system of claim 15, wherein the intent to prescribe calculation comprises five major components including a message affinity score, message frequency, message view time, channel potential, and frequency duration overlay.

17. The database and file management system of claim 15, wherein the predetermined range of the intent to prescribe score includes classifications as poor in a range from 1% to 25%, fair in a range from 26% to 50%, good in a range from 51% to 75%, and excellent in a range from 76% to 100%.

18. The database and file management system of claim 15, further comprising campaign measurable metrics scores are quotients of a following pair of scores.

19. The database and file management system of claim 15, further comprising a message frequency measurable metric which is a number of times a message is displayed.

20. The database and file management system of claim 15, further comprising a plurality of sensors coupled to the remote server configured to measure the measurable metrics.
